(19) United States
(12) Patent Application Publication
(10) Pub. No.: US 2024/0349608 A1
(43) Pub. Date: Oct. 17, 2024

(54) PHOTORESPONSIVE ASYMMETRIC NONFULLERENE ACCEPTORS OF THE A-D-A'-D-A TYPE FOR USE IN OPTOELECTRONIC DEVICES

(71) Applicant: Sumitomo Chemical Co., Ltd., Tokyo (JP)

(72) Inventors: Michal Maciejczyk, Godmanchester (GB); Nir Yaacobi-Gross, Godmanchester (GB)

(73) Assignee: Sumitomo Chemical Co., Ltd., Tokyo (JP)

(21) Appl. No.: 18/294,506

(22) PCT Filed: Aug. 5, 2022
(86) PCT No.: PCT/EP2022/072163

§ 371 Date: Feb. 1, 2024

(30) Foreign Application Priority Data
Aug. 6, 2021 (GB) 2111398.0
Mar. 24, 2022 (GB) 2204177.6
Mar. 24, 2022 (GB) 2204179.2
Jun. 7, 2022 (GB) 2208329.9

(51) Int. Cl. H10K 85/60; C07D 519/00; H10K 30/30

(52) U.S. Cl. CPC H10K 85/6576; C07D 519/00; H10K 85/626; H10K 85/655; H10K 85/657; H10K 30/30

(57) ABSTRACT

A compound of formula (I), A¹ is an electron-accepting group; D¹ and D² independently in each occurrence is an N electron-donating group; A¹, A² and A³ are each independently an electron-accepting group; B1 and B2 in each occurrence are independently a bridging group; x¹ and x² are each independently 0, 1, 2 or 3; y¹ and y² are each independently at least 1; z¹ and z² are each independently 0, 1, 2 or 3; and at least one of (i)-(iv) applies: (i) (D¹)y1 and (D²)y2 are different; (ii) A² and A³ are different; (iii) (B¹)x1 and (B¹)x2 are different; and (iv) (B²)z1 and (B²)z2 are different. The compound of formula (I) may be used as an electron acceptor in an organic photodetector.

### BACKGROUND

Embodiments of the present disclosure relate to electron-accepting compounds and more specifically, but not by way of limitation, to compounds containing electron-accepting and electron donating units, the compounds being suitable for use as an electron-accepting material in a photoresponsive device.

Electron-accepting non-fullerene compounds are known.

US20200328357 discloses an asymmetric A-D′-D-D″-A non-fullerene acceptor having two different π-bridges including alkylthienyl and alkoxythienyl units.

WO2020182174 discloses fluorescent compounds for bioimaging applications having a donor-acceptor (D-A) structure, in which the electron-donating and -withdrawing moieties are alternately arranged along a conjugated structure.

Zhang et al, “Non-Fullerene Acceptors with an Optical Response over 1000 nm toward Efficient Organic Solar Cells”, ACS Appl. Mater. Interfaces 2021, 13, 43, 51279-51288 discloses NFAs with π-bridge units and different end groups.

Kang et al, “Push-Pull Type Non-Fullerene Acceptors for Polymer Solar Cells: Effect of the Donor Core”, ACS Appl. Mater. Interfaces 2017, 9, 29, 24771-24777 discloses non-fullerene acceptors for solar cells.

Li et al, “Asymmetric A-D-n-A-type nonfullerene small molecule acceptors for efficient organic solar cells” J. Mater. Chem. A, 2019, 7, 19348-19354 discloses an asymmetric nonfullerene small molecule acceptor, TTPT-T-2F, with an A-D-n-A structure.

### SUMMARY

The present disclosure provides a compound of formula (I):

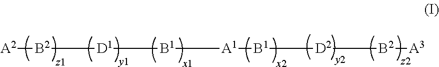

A¹ is an electron-accepting group.

D¹ and D² independently in each occurrence is an electron-donating group.

A¹, A² and A³ are each independently an electron-accepting group.

B¹ and B² in each occurrence are independently a bridging group.

x¹ and x² are each independently 0, 1, 2 or 3.

y¹ and y² are each independently at least 1.

z¹ and z² are each independently 0, 1, 2 or 3.

At least one of (i)-(iv) applies:
(D₁)_{y1} and (D²)_{y2} are different;
A² and A³ are different;
(B¹)ₓ₁ and (B¹)ₓ₂ are different; and
(B²)_{z1} and (B²)_{z2} are different.

The present disclosure provides a composition comprising an electron-donating material and an electron-accepting material wherein the electron accepting material is a compound as described herein.

The present disclosure provides an organic electronic device comprising an active layer comprising a compound or composition as described herein.

The present disclosure provides a photosensor comprising a light source and an organic photodetector as described herein, wherein the photosensor is configured to detect light emitted from the light source.

The present disclosure provides a formulation comprising a compound or composition as described herein dissolved or dispersed in one or more solvents.

The present disclosure provides a method of forming an organic electronic device as described herein wherein formation of the active layer comprises deposition of a formulation as described herein onto a surface and evaporation of the one or more solvents.

### DESCRIPTION OF DRAWINGS

The disclosed technology and accompanying figures describe some implementations of the disclosed technology.

FIG. 1 illustrates an organic photoresponsive device according to some embodiments.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

A compound of formula (I) as described herein may be provided in a bulk heterojunction layer of a photoresponsive device, preferably a photodetector, in which the bulk heterojunction layer is disposed between an anode and a cathode.

The bulk heterojunction layer comprises or consists of an electron-donating material and an electron-accepting compound of formula (I) as described herein.

In some embodiments, the bulk heterojunction layer contains two or more accepting materials and/or two or more electron-accepting materials.

In some embodiments, the weight of the electron-donating material(s) to the electron-accepting material(s) is from about 1:0.5 to about 1:2, preferably about 1:1.1 to about 1:2.

Preferably, the electron-donating material has a type II interface with the electron-accepting material, i.e. the electron-donating material has a shallower HOMO and LUMO that the corresponding HOMO and LUMO levels of the electron-accepting material. Preferably, the compound of formula (I) or (X) has a HOMO level that is at least 0.05 eV deeper, optionally at least 0.10 eV deeper, than the HOMO of the electron-donating material.

Optionally, the gap between the HOMO level of the electron-donating material and the LUMO level of the electron-accepting compound of formula (I) or (X) is less than 1.4 eV.

Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml) and spun at 3000 rpm directly on to the glassy carbon working electrode.

LUMO=4.8−E ferrocene (peak to peak average)−E reduction of sample (peak maximum).

HOMO=4.8−E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data.

In some embodiments, the compound of formula (I) has an absorption peak greater than 900 nm, optionally greater than 1000 nm.

Unless stated otherwise, absorption spectra of materials as described herein are measured using a Cary 5000 UV-VIS-NIR Spectrometer. Measurements were taken from 175 nm to 3300 nm using a PbSmart NIR detector for extended photometric range with variable slit widths (down to 0.01 nm) for optimum control over data resolution.

Absorption data are obtained by measuring the intensity of transmitted radiation through a solution sample. Absorption intensity is plotted vs. incident wavelength to generate an absorption spectrum. A method for measuring absorption may comprise measuring a 15 mg/ml solution in a quartz cuvette and comparing to a cuvette containing the solvent only.

Unless stated otherwise, absorption data as provided herein is as measured in toluene solution.

In some embodiments, the electron-accepting compound has formula (I):

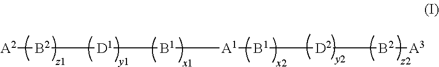

D¹ and D² independently in each occurrence is an electron-donating group.

A¹, A² and A³ are each independently an electron-accepting group.

B¹ and B² in each occurrence are independently a bridging group.

x¹ and x² are each independently 0, 1, 2 or 3, preferably 0 or 1.

y¹ and y² are each independently at least 1, preferably 1, 2 or 3, more preferably 1.

z¹ and z² are each independently 0, 1, 2 or 3, preferably 0 or 1.

—(B¹)ₓ₁-(D¹)_{y1}-(B²)_{z1}-A² is different from —(B¹)ₓ₂-(D²)_{y2}-(B²)_{z2}-A³. Such compounds are described hereinafter as “asymmetric” compounds.

At least one of (i)-(iv) applies:
(i) (D¹)_{y1} and (D²)_{y2} are different;
(ii) A² and A³ are different;
(iii) (B¹)ₓ₁ and (B¹)ₓ₂ are different; and
(iv) (B²)_{z1} and (B²)_{z2} are different.

In some preferred embodiments, D¹ and D² are different and y¹ and y² are the same or different.

In some preferred embodiments, y¹ and y² are different and D¹ and D² are the same or different.

In the case where (B¹)ₓ₁ is different from (B¹)ₓ₂ then either x¹ and x² are the same in which case B¹ of (B¹)ₓ₁ is different from B¹ of (B¹)ₓ₂, or x¹ and x² are different—preferably x¹ is 1 and x² is 0—in which case B¹ of (B¹)ₓ₁ is different from B¹ of (B¹)ₓ₂. Preferably, x¹ and x² are each 0.

In some preferred embodiments, (B²)_{z1} is different from (B²)_{z2}. According to these embodiments, either z¹ and z² are the same in which case B² of (B²)_{z1} is different from B² of (B²)_{z2}, or z¹ and z² are different—preferably z¹ is 1 and z² is 0—in which case B² of (B²)_{z1} is different from B² of (B²)_{z2}.

Optionally, B² of (B²)_{z1} is an optionally substituted monocyclic aromatic or heteroaromatic group and B² of (B²)_{z2} is a fused aromatic or heteroaromatic group.

A difference between D¹ and D², a difference between A² and A³, a difference between B¹ groups and a difference between B² groups may, in each case, be a difference in ring structure and/or a difference in substituents of rings.

In some embodiments, there is only one difference between —(B¹)ₓ₁-(D¹)_{y1}-(B²)_{z1}-A² and —(B¹)ₓ₂-(D²)_{y2}-(B²)_{z2}-A³ of formula (I).

In some embodiments, there are two or more differences between —(B¹)ₓ₁-(D¹)_{y1}-(B²)_{z1}-A² and —(B¹)ₓ₂-(D²)_{y2}-(B²)_{z2}-A³ of formula (I). In a preferred embodiment, y¹ and y² are both 1; D¹ and D² are different; and (B²)_{z1} is different from (B²)_{z2}.

Each of the electron-accepting groups A¹, A² and A³ has a lowest unoccupied molecular orbital (LUMO) level that is deeper (i.e., further from vacuum) than the LUMO of either of the electron-donating groups D¹ or D², preferably at least 1 eV deeper. The LUMO levels of electron-accepting groups and electron-donating groups may be as determined by modelling the LUMO level of these groups, in which each bond to adjacent group is replaced with a bond to a hydrogen atom. Modelling may be performed using Gaussian09 software available from Gaussian using Gaussian09 with B3LYP (functional) and LACVP* (Basis set).

In some embodiments, A¹ of formula (I) is a group of formula (II):

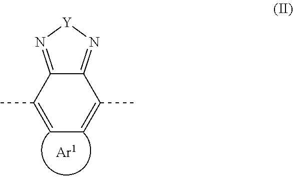

wherein:
Ar¹ is an aromatic or heteroaromatic group; and
Y is O, S, NR⁴ or R¹—C═C—R¹ wherein R¹ in each occurrence is independently H or a substituent wherein two substituents R¹ may be linked to form a monocyclic or polycyclic ring; and R⁴ is H or a substituent.

### Acceptor Unit A¹

In the case where A¹ is a group of formula (II), Ar¹ may be a monocyclic or polycyclic heteroaromatic group which is unsubstituted or substituted with one or more R² groups wherein R² in each occurrence is independently a substituent.

Preferred R² groups are selected from
F;
CN;
NO₂;
C₁₋₂₀ alkyl wherein one or more non-adjacent C atoms may be replaced with O, S, NR⁷ wherein R⁷ is a C₁₋₁₂ hydrocarbyl, COO or CO and one or more H atoms of the alkyl may be replaced with F;
an aromatic or heteroaromatic group, preferably phenyl, which is unsubstituted or substituted with one or more substituents; and
a group selected from

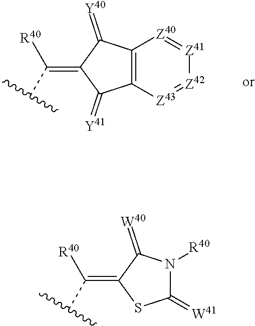

wherein Z⁴⁰, Z⁴¹, Z⁴² and Z⁴³ are each independently CR¹³ or N wherein R¹³ in each occurrence is H or a substituent, preferably a C₁₋₂₀ hydrocarbyl group; Y⁴⁰ and Y⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰; W⁴⁰ and W⁴¹ are each independently O, S, NX⁷¹ or CX⁶⁰X⁶¹ wherein X⁶⁰ and X⁶¹ is independently CN, CF₃ or COOR⁴⁰; and R⁴⁰ in each occurrence is H or a substituent, preferably H or a C₁₋₂₀ hydrocarbyl group. Exemplary substituents of an aromatic or heteroaromatic group R² are F, CN, NO₂, and C₁₋₁₂ alkyl wherein one or more non-adjacent C atoms may be replaced with O, S, NR⁷, COO or CO and one or more H atoms of the alkyl may be replaced with F.
R⁷ as described anywhere herein may be, for example, C₁₋₁₂ alkyl, unsubstituted phenyl; or phenyl substituted with one or more C₁₋₆ alkyl groups.

If a C atom of an alkyl group as described anywhere herein is replaced with another atom or group, the replaced C atom may be a terminal C atom of the alkyl group or a non-terminal C-atom.

By “non-terminal C atom” of an alkyl group as used anywhere herein means a C atom other than the C atom of the methyl group at the end of an n-alkyl chain or the C atoms of the methyl groups at the ends of a branched alkyl chain.

If a terminal C atom of a group as described anywhere herein is replaced then the resulting group may be an anionic group comprising a countercation, e.g., an ammonium or metal countercation, preferably an ammonium or alkali metal cation.

A C atom of an alkyl substituent group which is replaced with another atom or group as described anywhere herein is preferably a non-terminal C atom, and the resultant substituent group is preferably non-ionic.

Exemplary monocyclic heteroaromatic groups Ar¹ are oxadiazole, thiadiazole, triazole and 1,4-diazine which is unsubstituted or substituted with one or more substituents. Thiadiazole is particularly preferred.

Exemplary polycyclic heteroaromatic groups Ar¹ are groups of formula (V):

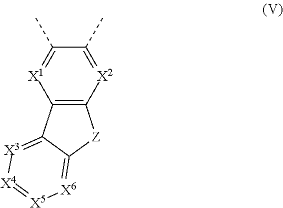

X¹ and X², are each independently selected from N and CR³ wherein R³ is H or a substituent, optionally H or a substituent R² as described above.

X³, X⁴, X⁵ and X⁶ are each independently selected from N and CR³ with the proviso that at least one of X³, X⁴, X⁵ and X⁶ is CR³.

Z is selected from O, S, SO₂, NR⁴, PR⁴, C(R³)₂, Si(R³)₂ C═O, C═S and C═C(R⁵)₂ wherein R³ is as described above; R⁴ is H or a substituent; and R⁵ in each occurrence is an electron-withdrawing group.

Optionally, each R⁴ of any NR⁴ or PR⁴ described anywhere herein is independently selected from H; C₁₋₂₀ alkyl wherein one or more non-adjacent C atoms other than the C atom bound to N or P may be replaced with O, S, NR⁷, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms of the alkyl may be replaced with O, S, NR⁷, COO or CO and one or more H atoms of the alkyl may be replaced with F.

Preferably, each R⁵ is CN, COOR⁴⁰; or CX⁶⁰X⁶¹ wherein X⁶⁰ and X⁶¹ is independently CN, CF₃ or COOR⁴⁰ and R⁴⁰ in each occurrence is H or a substituent, preferably H or a C₁₋₂₀ hydrocarbyl group.

A¹ groups of formula (II) are preferably selected from groups of formulae (IIa) and (IIb):

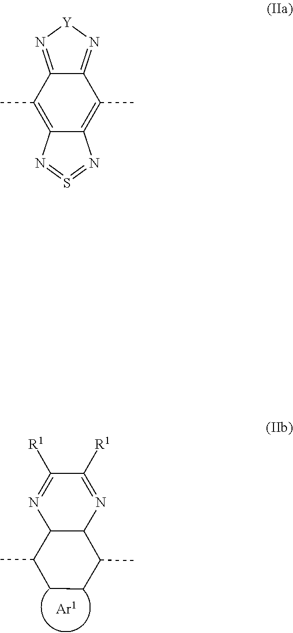

For compounds of formula (IIb), the two R¹ groups may or may not be linked.

Preferably, when the two R¹ groups are not linked each R¹ is independently selected from H; F; CN; NO₂; C₁₋₂₀ alkyl wherein one or more non-adjacent C atoms may be replaced with O, S, NR⁷, CO, COO, NR⁴, PR⁴, or Si(R³)₂ wherein R³ and R⁴ are as described above and one or more H atoms may be replaced with F; and aryl or heteroaryl, preferably phenyl, which may be unsubstituted or substituted with one or more substituents. Substituents of the aryl or heteroaryl group may be selected from one or more of F; CN; NO₂; and C₁₋₂₀ alkyl wherein one or more non-adjacent C atoms may be replaced with O, S, NR⁷, CO, COO and one or more H atoms may be replaced with F.

Preferably, when the two R¹ groups are linked, the group of formula (IIb) has formula (IIb-1) or (IIb-2):

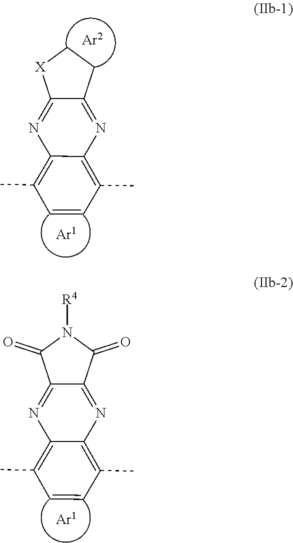

Ar² is an aromatic or heteroaromatic group, preferably benzene, which is unsubstituted or substituted with one or more substituents. Ar² may be unsubstituted or substituted with one or more substituents R² as described above.

X is selected from O, S, SO₂, NR⁴, PR⁴, C(R³)₂, Si(R³)₂ C═O, C═S and C═C(R⁵)₂ wherein R³, R⁴ and R⁵ are as described above.

Exemplary electron-accepting groups of formula (II) include, without limitation:

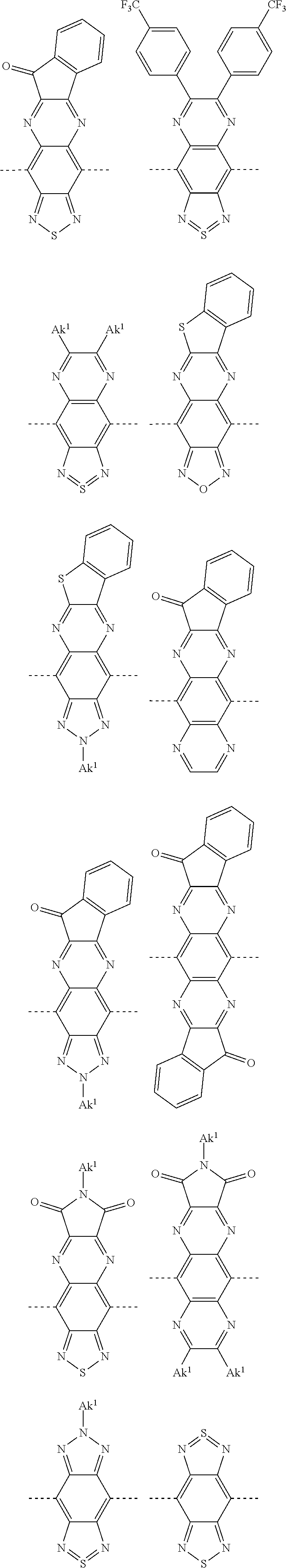

wherein Ak¹ is a C₁₋₂₀ alkyl group

Divalent electron-accepting groups other than formula (II) are optionally selected from formulae (IVa)-(IVj)

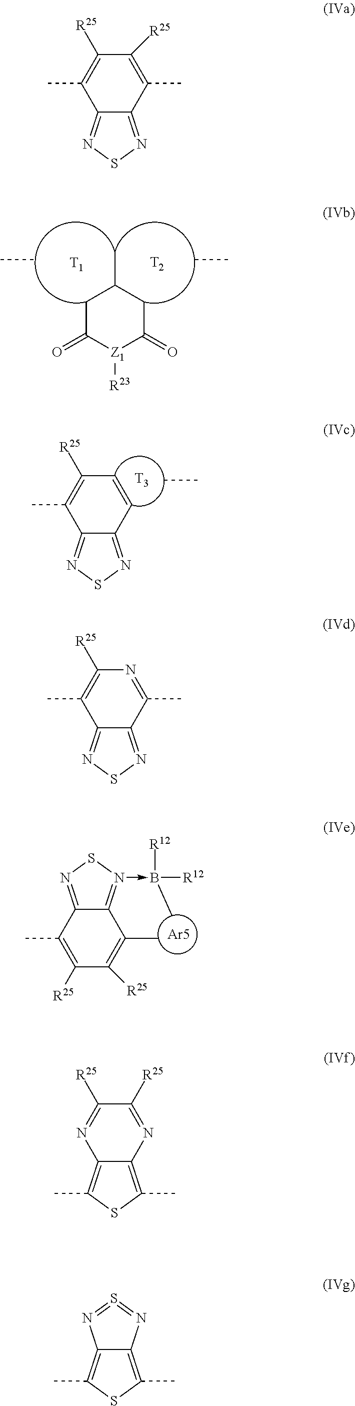

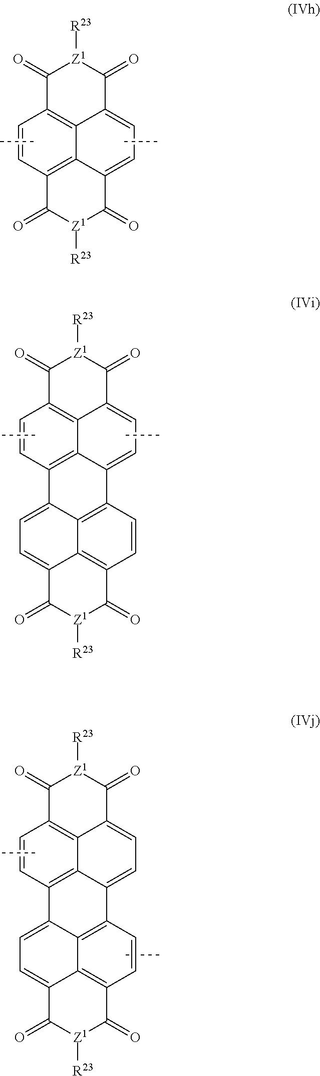

R²³ in each occurrence is a substituent, optionally C₁₋₁₂ alkyl wherein one or more non-adjacent C atoms other than the C atom attached to Z¹ may be replaced with O, S, NR⁷, COO or CO and one or more H atoms of the alkyl may be replaced with F.

R²⁵ in each occurrence is independently H; F; CN; NO₂; C₁₋₁₂ alkyl wherein one or more non-adjacent C atoms may be replaced with O, S, NR⁷, COO or CO and one or more H atoms of the alkyl may be replaced with F; an aromatic group, optionally phenyl, which is unsubstituted or substituted with one or more substituents selected from F and C₁₋₁₂ alkyl wherein one or more non-adjacent C atoms may be replaced with O, S, NR⁷, COO or CO; or

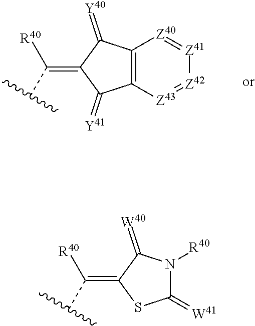

wherein Z⁴⁰, Z⁴¹, Z⁴² and Z⁴³ are each independently CR¹³ or N wherein R¹³ in each occurrence is H or a substituent, preferably a C₁₋₂₀ hydrocarbyl group;
Y⁴⁰ and Y⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰;
W⁴⁰ and W⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰; and
R⁴⁰ in each occurrence is H or a substituent, preferably H or a C₁₋₂₀ hydrocarbyl group.

Z¹ is N or P.

T¹, T² and T³ each independently represent an aryl or a heteroaryl ring, optionally benzene, which may be fused to one or more further rings. Substituents of T¹, T² and T³, where present, are optionally selected from non-H groups of R²⁵.

R¹² in each occurrence is a substituent, preferably a C₁₋₂₀ hydrocarbyl group.

Ar⁵ is an arylene or heteroarylene group, optionally thiophene, fluorene or phenylene, which may be unsubstituted or substituted with one or more substituents, optionally one or more non-H groups selected from R²⁵.

Electron-Accepting Groups A², A³

The monovalent acceptor Groups A² and A³ may each independently be selected from any such units known to the skilled person. A² and A³ may be the same or different, preferably different.

Exemplary monovalent acceptor units include, without limitation, units of formulae (IIIa)-(IIIq)

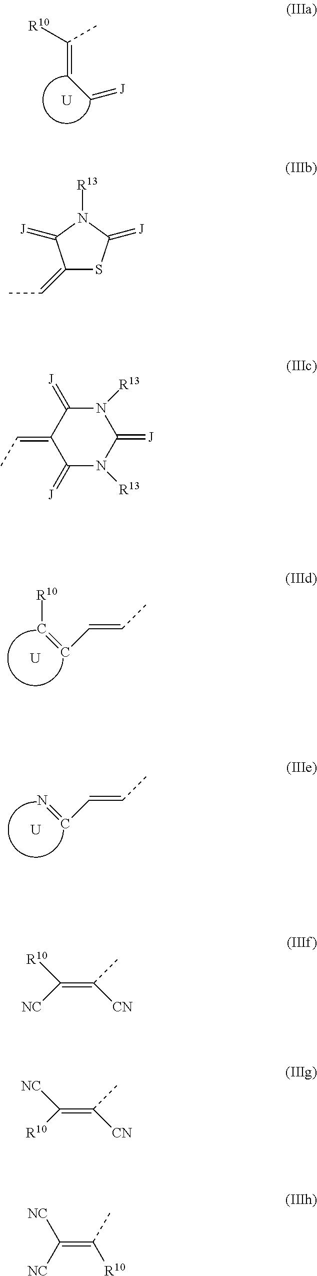

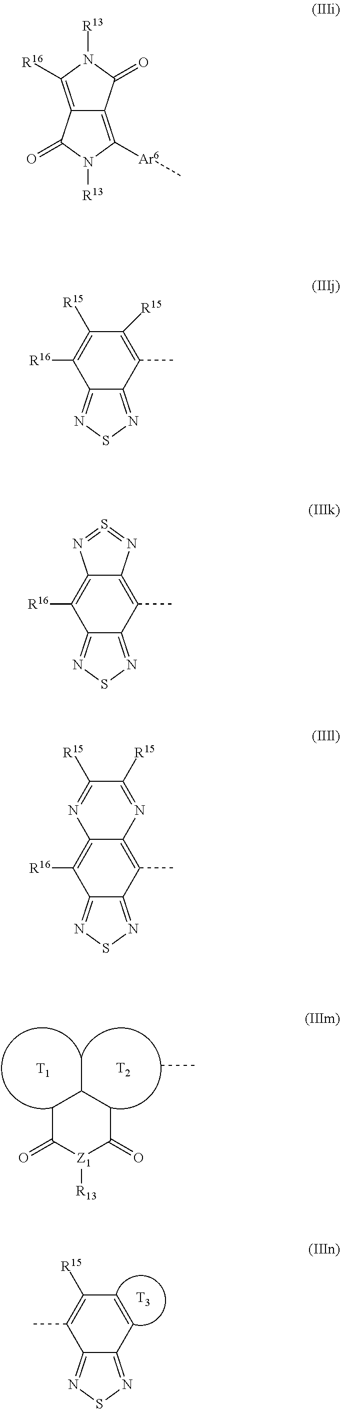

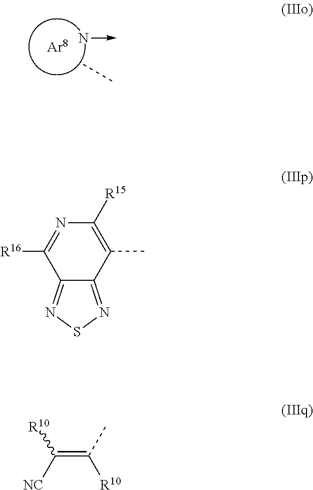

U is a 5- or 6-membered ring which is unsubstituted or substituted with one or more substituents and which may be fused to one or more further rings.

The N atom of formula (IIIe) may be unsubstituted or substituted.

R¹⁰ is H or a substituent, preferably a substituent selected from the group consisting of C₁₋₁₂ alkyl wherein one or more non-adjacent C atoms may be replaced with O, S, NR⁷, COO or CO and one or more H atoms of the alkyl may be replaced with F; and an aromatic group, optionally phenyl, which is unsubstituted or substituted with one or more substituents selected from F and C₁₋₁₂ alkyl wherein one or more non-adjacent C atoms may be replaced with O, S, NR⁷, COO or CO.

Preferably, R¹⁰ is H.

J is O or S, preferably O.

R¹³ in each occurrence is a substituent, optionally C₁₋₁₂ alkyl wherein one or more non-adjacent C atoms may be replaced with O, S, NR⁷, COO or CO and one or more H atoms of the alkyl may be replaced with F.

R¹⁵ in each occurrence is independently H; F; C₁₋₁₂ alkyl wherein one or more non-adjacent C atoms may be replaced with O, S, NR⁷, COO or CO and one or more H atoms of the alkyl may be replaced with F; aromatic group Ar², optionally phenyl, which is unsubstituted or substituted with one or more substituents selected from F and C₁₋₁₂ alkyl wherein one or more non-adjacent C atoms may be replaced with O, S, NR⁷, COO or CO; or a group selected from:

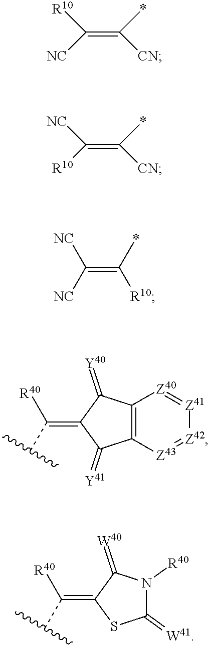

R¹⁶ is H or a substituent, preferably a substituent selected from:
—(Ar³)_{w} wherein Ar³ in each occurrence is independently an unsubstituted or substituted aryl or heteroaryl group, preferably thiophene, and w is 1, 2 or 3;

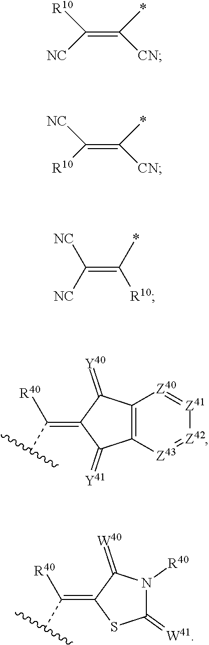

and
C₁₋₁₂ alkyl wherein one or more non-adjacent C atoms may be replaced with O, S, NR⁷, COO or CO and one or more H atoms of the alkyl may be replaced with F.

Ar⁶ is a 5-membered heteroaromatic group, preferably thiophene or furan, which is unsubstituted or substituted with one or more substituents.

Substituents of Ar³ and Ar⁶, where present, are optionally selected from C₁₋₁₂ alkyl wherein one or more non-adjacent C atoms may be replaced with O, S, NR⁷, COO or CO and one or more H atoms of the alkyl may be replaced with F.

T¹, T² and T³ are each independently as described above.

Ar⁸ is a fused heteroaromatic group which is unsubstituted or substituted with one or more substituents, optionally one or more non-H substituents R¹⁰, and which is bound to an aromatic C atom of B² and to a boron substituent of B².

Preferred groups A² and A³ are groups having a non-aromatic carbon-carbon bond which is bound directly to D1 or D2 or, if present to B².

Preferably at least one of A² and A³, preferably both of A² and A³, are a group of formula (IIIa-1):

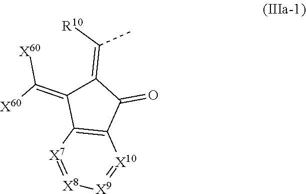

wherein:
R¹⁰ is as described above;
each X⁷-X¹⁰ is independently CR¹² or N wherein R¹² in each occurrence is H or a substituent selected from C₁₋₂₀ hydrocarbyl and an electron withdrawing group. Preferably, the electron withdrawing group is F, Cl, Br or CN, more preferably F, Cl or CN; and
X⁶⁰ and X⁶¹ is independently CN, CF₃ or COOR⁴⁰ wherein R⁴⁰ in each occurrence is H or a substituent, preferably H or a C₁₋₂₀ hydrocarbyl group. Preferably, X⁶⁰ and X⁶¹ are each CN.

The C₁₋₂₀ hydrocarbyl group R¹² may be selected from C₁₋₂₀ alkyl; unsubstituted phenyl; and phenyl substituted with one or more C₁₋₁₂ alkyl groups.

Exemplary groups of formula (IIId) include:

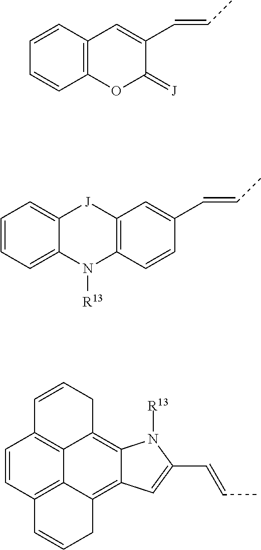

Exemplary groups of formula (IIIe) include:

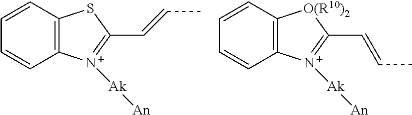

An exemplary group of formula (IIIq) is:

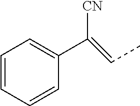

An exemplary group of formula (IIIg) is:

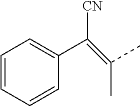

An exemplary group of formula (IIIj) is:

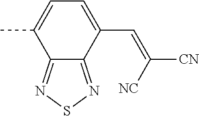

wherein Ak is a C₁₋₁₂ alkylene chain in which one or more C atoms may be replaced with O, S, NR⁷, CO or COO; An is an anion, optionally —SO₃⁻; and each benzene ring is independently unsubstituted or substituted with one or more substituents selected from substituents described with reference to R¹⁰.

Exemplary groups of formula (IIIm) are:

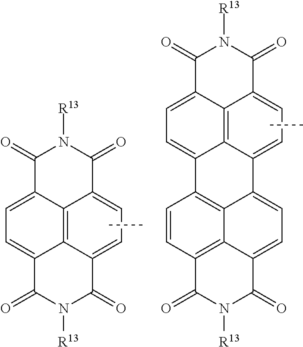

An exemplary group of formula (IIIn) is:

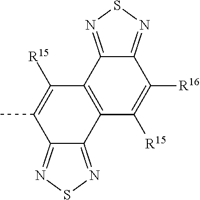

Groups of formula (IIIo) are bound directly to a bridging group B² substituted with a —B(R¹⁴)₂ wherein R¹⁴ in each occurrence is a substituent, optionally a C₁₋₂₀ hydrocarbyl group; → is a bond to the boron atom —B(R¹⁴)₂ of R³ or R⁶; and --- is the bond to B².

Optionally, R¹⁴ is selected from C₁₋₁₂ alkyl; unsubstituted phenyl; and phenyl substituted with one or more C₁₋₁₂ alkyl groups.

The group of formula (IIIo), the B² group and the B(R¹⁴)₂ substituent of B² may be linked together to form a 5- or 6-membered ring.

Optionally groups of formula (IIIo) are selected from:

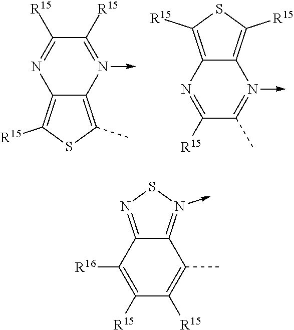

### Bridging Units

Bridging units B¹ and B² are preferably each selected from vinylene, arylene, heteroarylene, arylenevinylene and heteroarylenevinylene wherein the arylene and heteroarylene groups are monocyclic or bicyclic groups, each of which may be unsubstituted or substituted with one or more substituents.

Optionally, B¹ and B² are is selected from units of formulae (VIa)-(VIn):

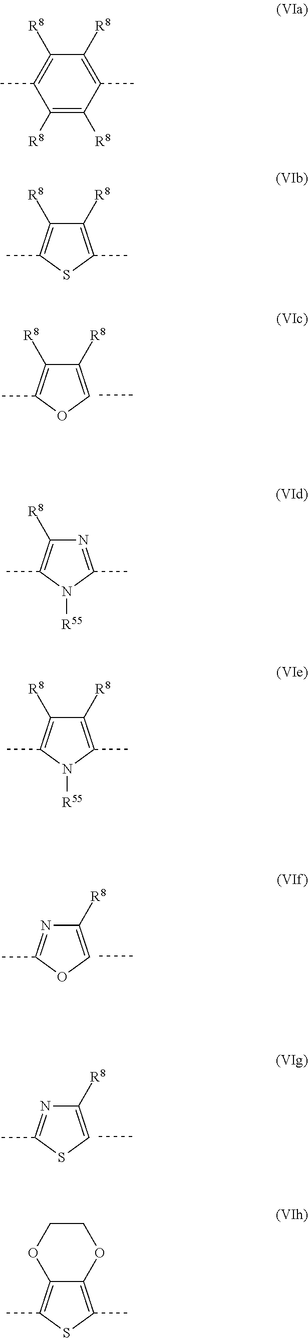

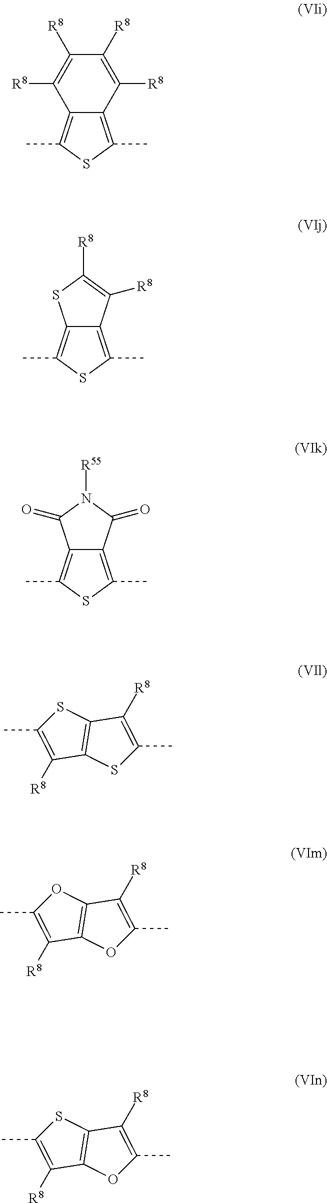

wherein Y^{A} is O, S or NR⁵⁵ wherein R⁵⁵ is H or a substituent; R⁸ in each occurrence is independently H or a substituent, preferably H or a substituent selected from F; CN; NO₂; C₁₋₂₀ alkyl wherein one or more non-adjacent C atoms may be replaced with O, S, NR⁷, COO or CO and one or more H atoms of the alkyl may be replaced with F; phenyl which is unsubstituted or substituted with one or more substituents; and —B(R¹⁴)₂ wherein R¹⁴ in each occurrence is a substituent, optionally a C₁₋₂₀ hydrocarbyl group. R⁸ groups of formulae (VIa), (VIb) and (VIc) may be linked to form a bicyclic ring, for example thienopyrazine.

R⁸ is preferably H, C₁₋₂₀ alkyl or C₁₋₁₉ alkoxy.

Electron-Donating Groups D¹ and D²

Electron-donating groups preferably are fused aromatic or heteroaromatic groups, more preferably fused heteroaromatic groups containing 3 or more rings. Particularly preferred electron-donating groups comprise fused thiophene or furan rings, optionally fused rings containing thiophene or furan rings and one or more rings selected from benzene, cyclopentadiene, tetrahydropyran, tetrahydrothiopyran and piperidine rings, each of said rings being unsubstituted or substituted with one or more substituents.

Exemplary electron-donating groups D¹ and D² include groups of formulae (VIIa)-(VIIp):

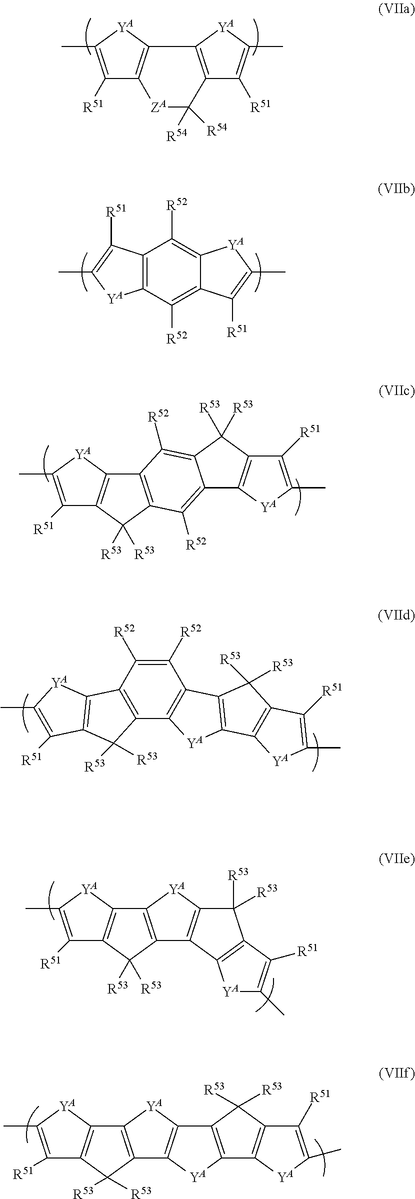

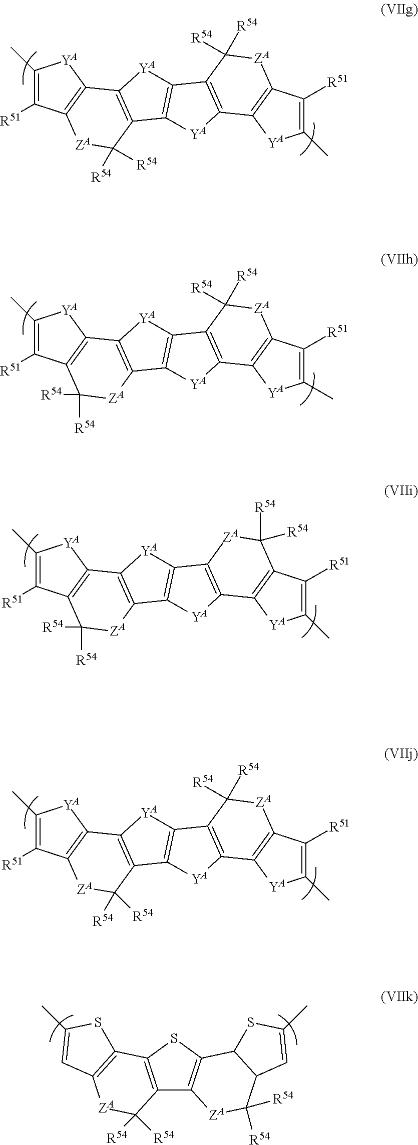

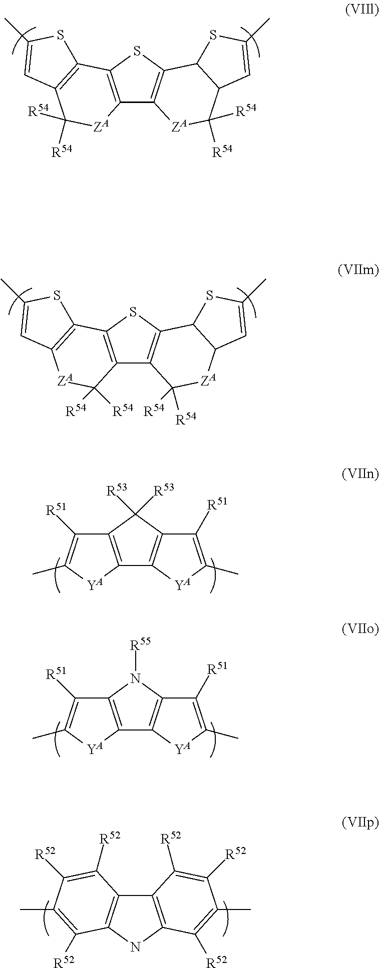

wherein Y^{A} in each occurrence is independently O, S or NR⁵⁵, Z^{A} in each occurrence is O, CO, S, NR⁵⁵ or C(R⁵⁴)₂; R⁵¹, R⁵² R⁵⁴ and R⁵⁵ independently in each occurrence is H or a substituent; and R⁵³ independently in each occurrence is a substituent.

Optionally, R⁵¹ and R⁵² independently in each occurrence are selected from H; F; C₁₋₂₀ alkyl wherein one or more non-adjacent C atoms may be replaced with O, S, NR⁷, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g., phenyl.

The one or more substituents of Ar³, if present, may be selected from C₁₋₁₂ alkyl wherein one or more non-adjacent C atoms may be replaced with O, S, NR⁷, COO or CO and one or more H atoms of the alkyl may be replaced with F.

Preferably, each R⁵⁴ is selected from the group consisting of:
H;
F;
linear, branched or cyclic C₁₋₂₀ alkyl wherein one or more non-adjacent C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula (Ak)u-(Ar⁷)v wherein Ak is a C₁₋₂₀ alkylene chain in which one or more non-adjacent C atoms may be replaced with O, S, NR⁷, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₂₀ alkyl wherein one or more non-adjacent C atoms may be replaced with O, S, NR⁷, CO or COO and one or more H atoms may be replaced with F. Preferably, Ar⁷ is phenyl.

Preferably, each R⁵¹ is H.

Optionally, R⁵³ independently in each occurrence is selected from C₁₋₂₀ alkyl wherein one or more non-adjacent C atoms may be replaced with O, S, NR⁷, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁷, COO or CO and one or more H atoms of the alkyl may be replaced with F.

Preferably, R⁵⁵ as described anywhere herein is H or C₁₋₃₀ hydrocarbyl group.

Preferably, D¹ and D² are each independently a group of formula (VIIa). Exemplary groups of formula (VIIa) include, without limitation:

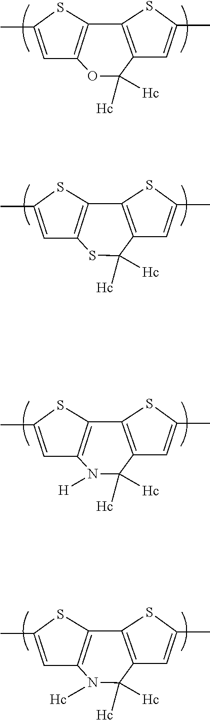

wherein He in each occurrence is independently a C₁₋₂₀ hydrocarbyl group, e.g., C₁₋₂₀ alkyl, unsubstituted aryl, or aryl substituted with one or more C₁₋₁₂ alkyl groups. The aryl group is preferably phenyl.

In some embodiments, y¹ and y² are each 1.

In some embodiments, at least one of y¹ and y² is greater than 1. In these embodiments, the chain of D¹ and/or D² groups, respectively, may be linked in any orientation. For example, in the case where D¹ is a group of formula (VIIa) and y¹ is 2, -[D¹]_{y1}—may be selected from any of:

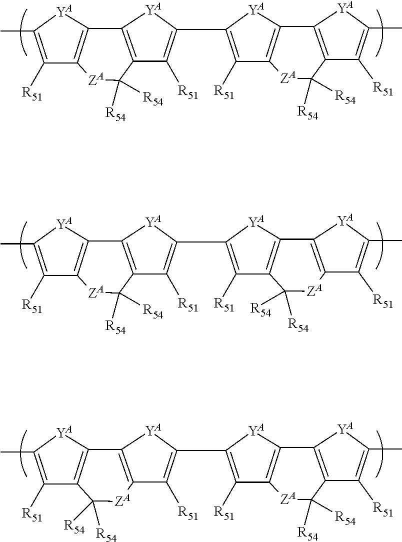

### Electron-Donating Material

A bulk heterojunction layer as described herein comprises an electron-donating material and a compound of formula (I) or (X) as described herein.

Exemplary donor materials are disclosed in, for example, WO2013051676, the contents of which are incorporated herein by reference.

The electron-donating material may be a non-polymeric or polymeric material.

In a preferred embodiment the electron-donating material is an organic conjugated polymer, which can be a homopolymer or copolymer including alternating, random or block copolymers. The conjugated polymer is preferably a donor-acceptor polymer comprising alternating electron-donating repeat units and electron-accepting repeat units.

Preferred are non-crystalline or semi-crystalline conjugated organic polymers.

Further preferably the electron-donating polymer is a conjugated organic polymer with a low bandgap, typically between 2.5 eV and 1.5 eV, preferably between 2.3 eV and 1.8 eV. Optionally, the electron-donating polymer has a HOMO level no more than 5.5 eV from vacuum level. Optionally, the electron-donating polymer has a HOMO level at least 4.1 eV from vacuum level. As exemplary electron-donating polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4-bisubstituted selenophene), poly(bisthiophene), poly(terthiophene), poly(bisselenophene), poly(terselenophene), polythieno[2,3-b]thiophene, polythieno[3,2-b]thiophene, polybenzothiophene, polybenzo[1,2-b:4,5-b′jdithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned.

Preferred examples of donor polymers are copolymers of polyfluorenes and polythiophenes, each of which may be substituted, and polymers comprising benzothiadiazole-based and thiophene-based repeating units, each of which may be substituted.

A particularly preferred donor polymer comprises donor unit (VIIa) provided as a repeat unit of the polymer, most preferably with an electron-accepting repeat unit, for example divalent electron-accepting units as described herein provided as polymeric repeat units.

### Additional Electron-Accepting Materials

In some embodiments, the compound of formula (I) or (X) as described herein is the only electron-accepting material of a bulk heterojunction layer.

In some embodiments, the bulk heterojunction layer contains a compound of formula (I) or (X) and one or more further electron-accepting materials. The one or more further electron-accepting materials may be selected from non-fullerene acceptors and fullerenes.

Non-fullerene acceptors are described in, for example, Cheng et. al., “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g., fluorinated derivatives thereof such as ITIC-4F and IEICO-4F.

Exemplary fullerene electron-accepting compounds are C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g. tolyl-C₆₁-butyric acid methyl ester (C₆₀TCBM)), and ThCBM-type fullerene derivatives (e.g. thienyl-C₆₁-butyric acid methyl ester (C₆₀ThCBM).

Fullerene derivatives may have formula (V):

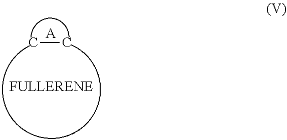

wherein A, together with the C—C group of the fullerene, forms a monocyclic or fused ring group which may be unsubstituted or substituted with one or more substituents.

Exemplary fullerene derivatives include formulae (Va), (Vb) and (Vc):

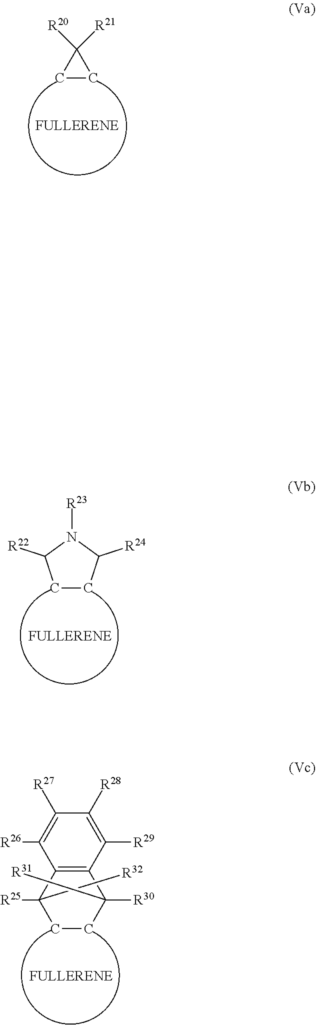

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent C atoms may be replaced with O, S, NR⁷, CO or COO and one or more H atoms may be replaced with F.

Substituents of aryl or heteroaryl, where present, are optionally selected from C₁₋₁₂ alkyl wherein one or more non-adjacent C atoms may be replaced with O, S, NR⁷, CO or COO and one or more H atoms may be replaced with F.

### Formulations

The bulk heterojunction layer may be formed by any process including, without limitation, thermal evaporation and solution deposition methods.

Preferably, the bulk heterojunction layer is formed by depositing a formulation comprising the electron-donating material(s), the electron-accepting material(s) and any other components of the bulk heterojunction layer dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron-accepting material, the electron-donating material and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

### Organic Electronic Device

A polymer or composition as described herein may be provided as an active layer of an organic electronic device. In a preferred embodiment, a bulk heterojunction layer of an organic photoresponsive device, more preferably an organic photodetector, comprises a composition as described herein.

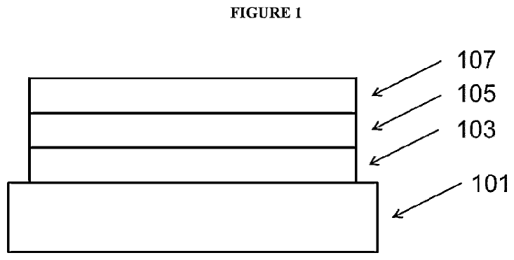

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent. The transmittance of a transparent electrode may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer. In some embodiments, a work function modification layer is disposed between the bulk heterojunction layer and the anode, and/or between the bulk heterojunction layer and the cathode.

The area of the OPD may be less than about 3 cm², less than about 2 cm², less than about 1 cm 2, less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer contains a polymer as described herein and an electron-accepting compound. The bulk heterojunction layer may consist of these materials or may comprise one or more further materials, for example one or more further electron-donating materials and/or one or more further electron-accepting compounds.

### Applications

A circuit may comprise the OPD connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm or at least 1000 nm, optionally in the range of 1000-1500 nm.

The present inventors have found that a material comprising an electron-accepting unit of formula (I) may be used for the detection of light at longer wavelengths, particularly 1300-1400 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g., due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

### Compound Example 1

Compound Example 1 was prepared according to the following reaction scheme:

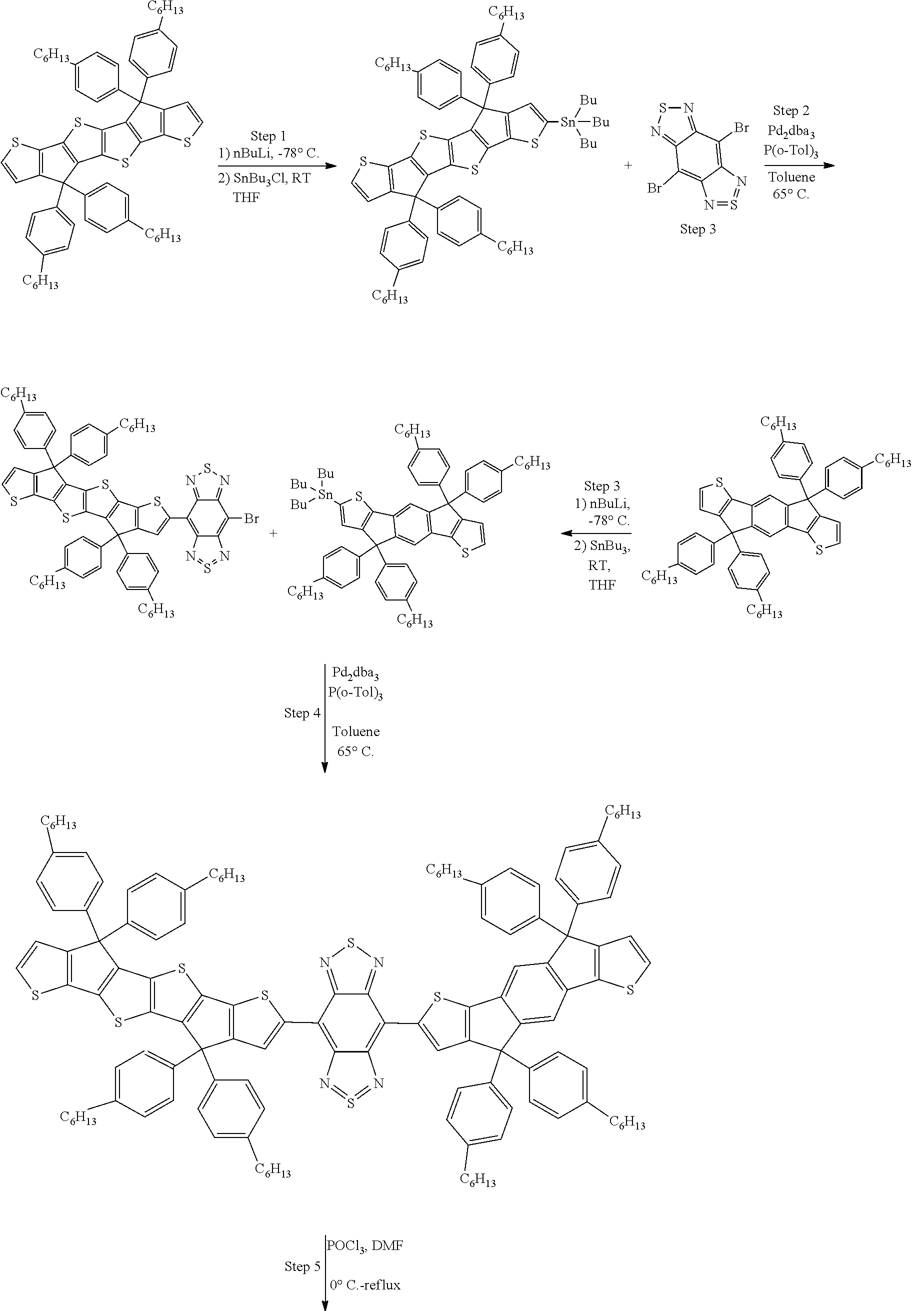

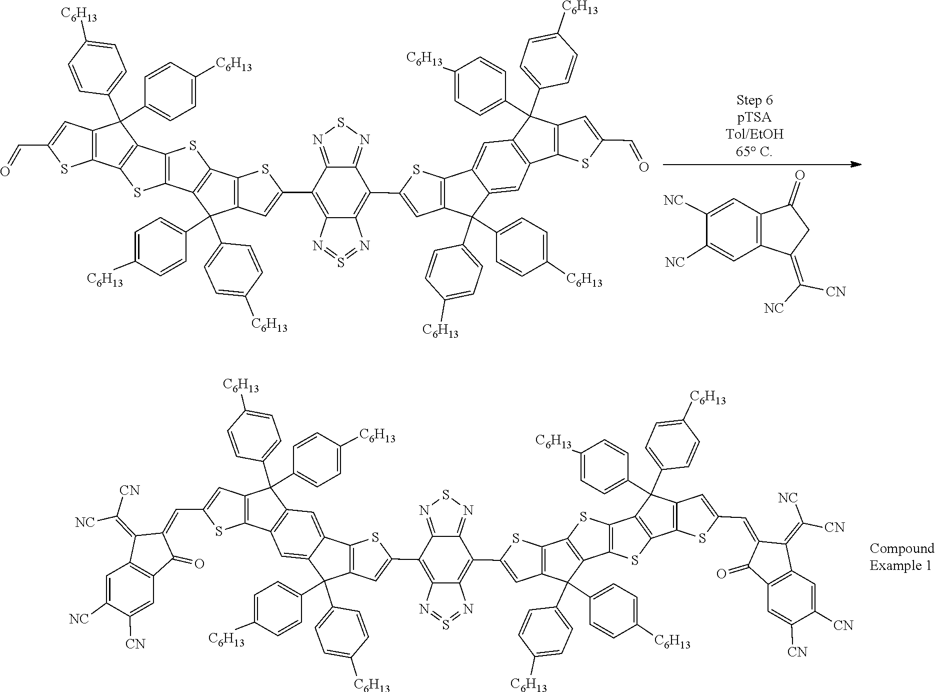

### Modelling Data

All modelling as described in these examples was performed using Gaussian09 software available from Gaussian using Gaussian09 with B3LYP (functional).

HOMO and LUMO levels were modelled for individual donor and acceptor units. Results are set out in Tables 2-4

TABLE 1

| Donor units D¹, D² | HOMO/eV | LUMO/eV |
|---|---|---|
| 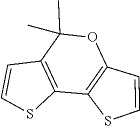 | −5.08 | −1.09 |
| 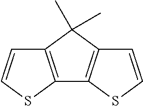 | −5.18 | −1.01 |
| 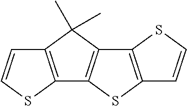 [SMILES: CC1(C)c2ccsc2-c2sc3ccsc3c21] | −5.06 | −1.25 |
| 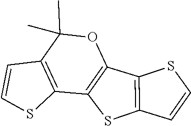 [SMILES: CC1(C)Oc2c(sc3ccsc23)-c2sccc21] | −4.97 | −1.32 |
| 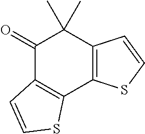 | −5.74 | −1.87 |
| 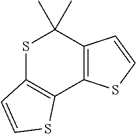 | −5.27 | −1.33 |

TABLE 2

| Acceptor units A², A³ | HOMO/eV | LUMO/eV |
|---|---|---|
| 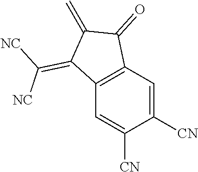 [SMILES: C=C1C(=O)c2cc(C#N)c(C#N)cc2C1=C(C#N)C#N] | −7.95 | −4.23 |
| 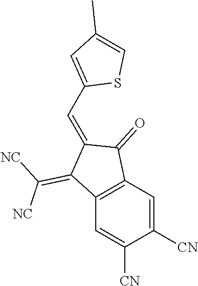 [SMILES: Cc1csc(/C=C2\C(=O)c3cc(C#N)c(C#N)cc3C2=C(C#N)C#N)c1] | −6.32 | −3.34 |
| 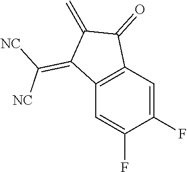 [SMILES: C=C1C(=O)c2cc(F)c(F)cc2C1=C(C#N)C#N] | −7.39 | −3.57 |

TABLE 3

| Acceptor Unit A¹ | HOMO/eV | LUMO/eV |
|---|---|---|
| 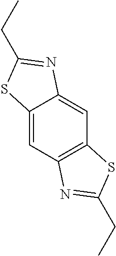 [SMILES: CCc1nc2cc3sc(CC)nc3cc2s1] | −6.02 | −1.25 |
| 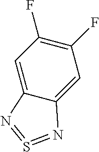 [SMILES: Fc1cc2c(cc1F)N=S=N2] | −6.95 | −2.60 |
| 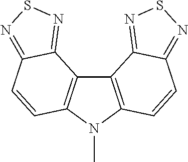 [SMILES: Cn1c2ccc3nsnc3c2c2c3nsnc3ccc21] | −5.72 | −2.15 |
| 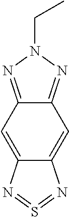 | −5.80 | −2.82 |
| 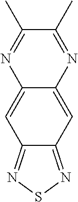 | −6.17 | −2.93 |
| 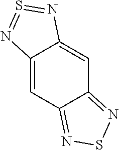 [SMILES: c1c2c(cc3nsnc13)N=S=N2] | −6.20 | −3.56 |
| 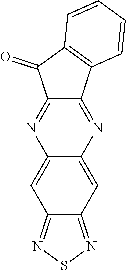 [SMILES: O=C1c2ccccc2-c2nc3cc4nsnc4cc3nc21] | −6.44 | −3.36 |

Acceptor units A¹ preferably have a modelled LUMO of at least 2.9 eV or at least 3.0 eV from vacuum level.

HOMO and LUMO levels were modelled for asymmetric compounds of formula (I) and comparative symmetric compounds. Results are set out in Tables 4-8 in which Sif corresponds to oscillator strength of the transition from Si (predicting absorption intensity), and Eopt is the modelled optical gap.

TABLE 4
Compounds in which D¹ and D² are different.

| | HOMO/ | LUMO/ | | | | Dipole |
|---|---|---|---|---|---|---|
| Structure/ID | eV | eV | Eg/nm | S1f | Eopt/nm | moment |
| 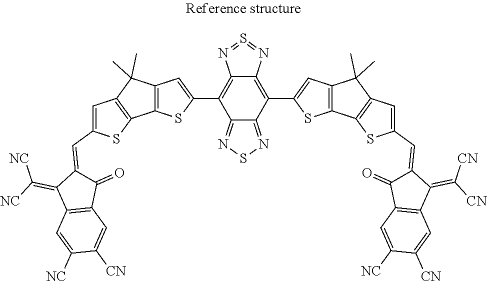 [SMILES: CC1(C)c2cc(/C=C3\C(=O)c4cc(C#N)c(C#N)cc4C3=C(C#N)C#N)sc2-c2sc(-c3c4c(c(-c5cc6c(s5)-c5sc(/C=C7\C(=O)c8cc(C#N)c(C#N)cc8C7=C(C#N)C#N)cc5C6(C)C)c5nsnc35)N=S=N4)cc21] | −5.63 | −4.57 | 1173 | 1.60 | 1209 | 16.3 |
| 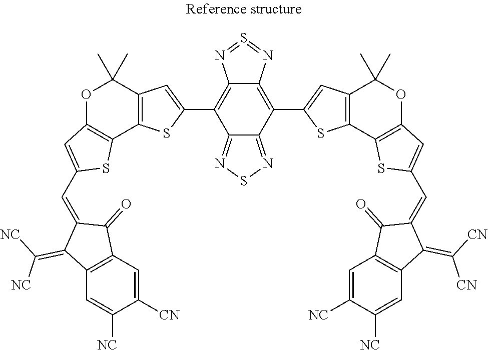 [SMILES: CC1(C)Oc2cc(/C=C3\C(=O)c4cc(C#N)c(C#N)cc4C3=C(C#N)C#N)sc2-c2sc(-c3c4c(c(-c5cc6c(s5)-c5sc(/C=C7\C(=O)c8cc(C#N)c(C#N)cc8C7=C(C#N)C#N)cc5OC6(C)C)c5nsnc35)N=S=N4)cc21] | −5.55 | −4.55 | 1237 | 1.36 | 1269 | 21.6 |
| 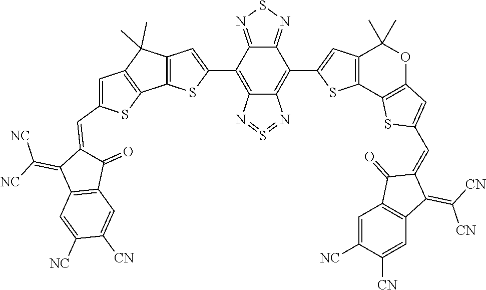 [SMILES: CC1(C)Oc2cc(/C=C3\C(=O)c4cc(C#N)c(C#N)cc4C3=C(C#N)C#N)sc2-c2sc(-c3c4c(c(-c5cc6c(s5)-c5sc(/C=C7\C(=O)c8cc(C#N)c(C#N)cc8C7=C(C#N)C#N)cc5C6(C)C)c5nsnc35)N=S=N4)cc21] | −5.59 | −4.56 | 1206 | 1.48 | 1239 | 19.2 |
| 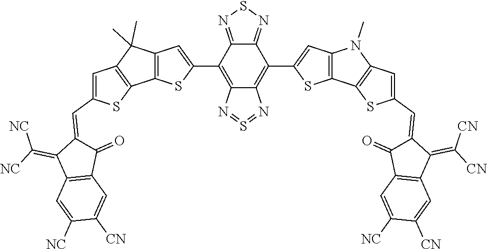 [SMILES: Cn1c2cc(/C=C3\C(=O)c4cc(C#N)c(C#N)cc4C3=C(C#N)C#N)sc2c2sc(-c3c4c(c(-c5cc6c(s5)-c5sc(/C=C7\C(=O)c8cc(C#N)c(C#N)cc8C7=C(C#N)C#N)cc5C6(C)C)c5nsnc35)N=S=N4)cc21] | −5.62 | −4.55 | 1162 | 1.62 | 1201 | 16.4 |
| 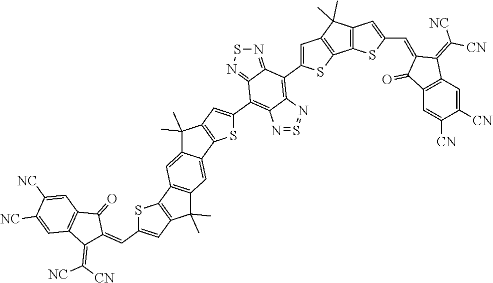 [SMILES: CC1(C)c2cc3c(cc2-c2sc(/C=C4\C(=O)c5cc(C#N)c(C#N)cc5C4=C(C#N)C#N)cc21)C(C)(C)c1cc(-c2c4c(c(-c5cc6c(s5)-c5sc(/C=C7\C(=O)c8cc(C#N)c(C#N)cc8C7=C(C#N)C#N)cc5C6(C)C)c5nsnc25)N=S=N4)sc1-3] | −5.47 | −4.37 | 1136 | 1.73 | 1216 | 7.48 |

TABLE 5
compounds in which z¹ is 1 and z² is 0

| | HOMO/ | LUMO/ | | | | Dipole |
|---|---|---|---|---|---|---|
| Structure/ID | eV | eV | Eg/nm | S1f | Eopt/nm | moment |
| 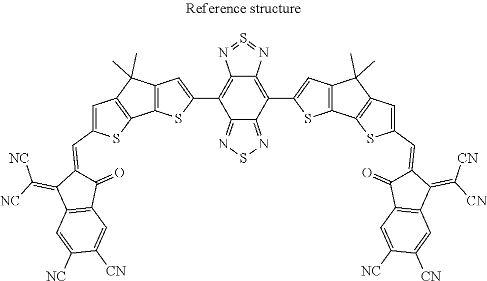 [SMILES: CC1(C)c2cc(/C=C3\C(=O)c4cc(C#N)c(C#N)cc4C3=C(C#N)C#N)sc2-c2sc(-c3c4c(c(-c5cc6c(s5)-c5sc(/C=C7\C(=O)c8cc(C#N)c(C#N)cc8C7=C(C#N)C#N)cc5C6(C)C)c5nsnc35)N=S=N4)cc21] | −5.63 | −4.57 | 1173 | 1.60 | 1209 | 16.3 |
| 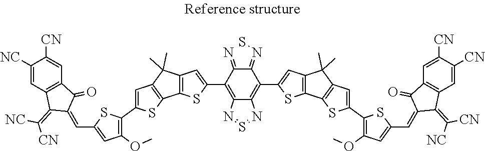 [SMILES: COc1cc(/C=C2\C(=O)c3cc(C#N)c(C#N)cc3C2=C(C#N)C#N)sc1-c1cc2c(s1)-c1sc(-c3c4c(c(-c5cc6c(s5)-c5sc(-c7sc(/C=C8\C(=O)c9cc(C#N)c(C#N)cc9C8=C(C#N)C#N)cc7OC)cc5C6(C)C)c5nsnc35)N=S=N4)cc1C2(C)C] | −5.10 | −4.23 | 1424 | 2.32 | 1430 | 5.97 |
| 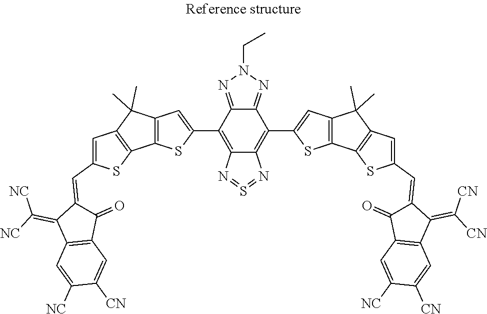 [SMILES: CCn1nc2c(-c3cc4c(s3)-c3sc(/C=C5\C(=O)c6cc(C#N)c(C#N)cc6C5=C(C#N)C#N)cc3C4(C)C)c3c(c(-c4cc5c(s4)-c4sc(/C=C6\C(=O)c7cc(C#N)c(C#N)cc7C6=C(C#N)C#N)cc4C5(C)C)c2n1)N=S=N3] | −5.56 | −4.27 | 962 | 2.51 | 955 | 19.18 |
| 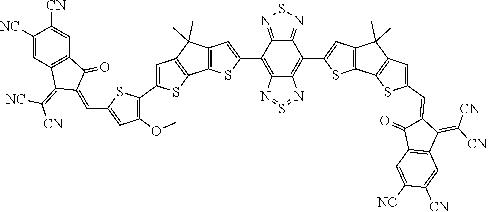 [SMILES: COc1cc(/C=C2\C(=O)c3cc(C#N)c(C#N)cc3C2=C(C#N)C#N)sc1-c1cc2c(s1)-c1sc(-c3c4c(c(-c5cc6c(s5)-c5sc(/C=C7\C(=O)c8cc(C#N)c(C#N)cc8C7=C(C#N)C#N)cc5C6(C)C)c5nsnc35)N=S=N4)cc1C2(C)C] | −5.34 | −4.38 | 1294 | 1.99 | 1311 | 7.00 |
| 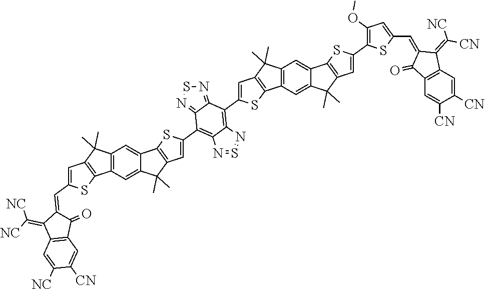 [SMILES: COc1cc(/C=C2\C(=O)c3cc(C#N)c(C#N)cc3C2=C(C#N)C#N)sc1-c1cc2c(s1)-c1cc3c(cc1C2(C)C)-c1sc(-c2c4c(c(-c5cc6c(s5)-c5cc7c(cc5C6(C)C)-c5sc(/C=C6\C(=O)c8cc(C#N)c(C#N)cc8C6=C(C#N)C#N)cc5C7(C)C)c5nsnc25)N=S=N4)cc1C3(C)C] | −5.13 | −4.06 | 1152 | 1.89 | 1283 | 10.4 |
| 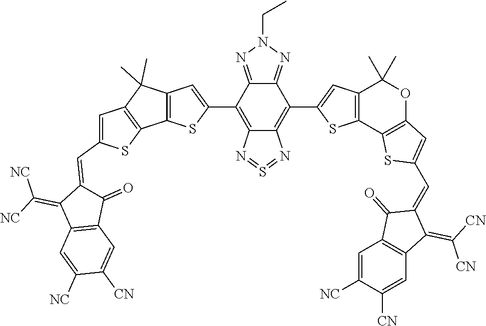 [SMILES: CCn1nc2c(-c3cc4c(s3)-c3sc(/C=C5\C(=O)c6cc(C#N)c(C#N)cc6C5=C(C#N)C#N)cc3OC4(C)C)c3c(c(-c4cc5c(s4)-c4sc(/C=C6\C(=O)c7cc(C#N)c(C#N)cc7C6=C(C#N)C#N)cc4C5(C)C)c2n1)N=S=N3] | −5.53 | −4.26 | 985 | 2.30 | 1020 | 22.3 |
| 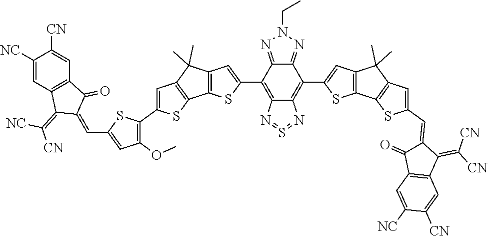 [SMILES: CCn1nc2c(-c3cc4c(s3)-c3sc(/C=C5\C(=O)c6cc(C#N)c(C#N)cc6C5=C(C#N)C#N)cc3C4(C)C)c3c(c(-c4cc5c(s4)-c4sc(-c6sc(/C=C7\C(=O)c8cc(C#N)c(C#N)cc8C7=C(C#N)C#N)cc6OC)cc4C5(C)C)c2n1)N=S=N3] | −5.29 | −4.11 | 1061 | 3.09 | 1088 | 8.7 |

TABLE 6
different B² groups

| | HOMO/ | LUMO/ | | | | Dipole |
|---|---|---|---|---|---|---|
| Structure/ID | eV | eV | Eg/nm | S1f | Eopt/nm | moment |
| 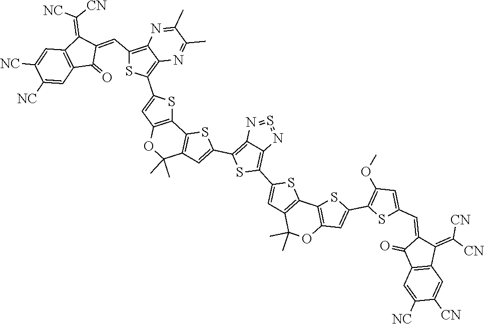 [SMILES: COc1cc(/C=C2\C(=O)c3cc(C#N)c(C#N)cc3C2=C(C#N)C#N)sc1-c1cc2c(s1)-c1sc(-c3sc(-c4cc5c(s4)-c4sc(-c6sc(/C=C7\C(=O)c8cc(C#N)c(C#N)cc8C7=C(C#N)C#N)c7nc(C)c(C)nc67)cc4OC5(C)C)c4c3N=S=N4)cc1C(C)(C)O2] | −5.11 | −4.11 | 1247 | | | |

TABLE 7
D¹ and D² are different and z¹ is 1 and z² is 0

| | HOMO/ | LUMO/ | | | | Dipole |
|---|---|---|---|---|---|---|
| Structure/ID | eV | eV | Eg/nm | S1f | Eopt/nm | moment |
| 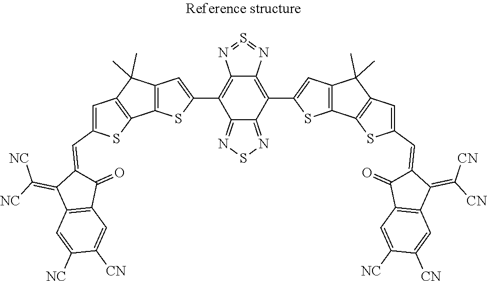 [SMILES: CC1(C)c2cc(/C=C3\C(=O)c4cc(C#N)c(C#N)cc4C3=C(C#N)C#N)sc2-c2sc(-c3c4c(c(-c5cc6c(s5)-c5sc(/C=C7\C(=O)c8cc(C#N)c(C#N)cc8C7=C(C#N)C#N)cc5C6(C)C)c5nsnc35)N=S=N4)cc21] | −5.63 | −4.57 | 1173 | 1.60 | 1209 | 16.3 |
| 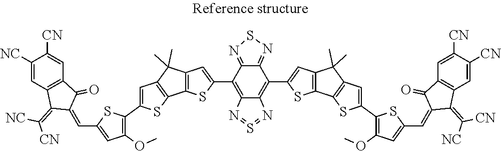 [SMILES: COc1cc(/C=C2\C(=O)c3cc(C#N)c(C#N)cc3C2=C(C#N)C#N)sc1-c1cc2c(s1)-c1sc(-c3c4c(c(-c5cc6c(s5)-c5sc(-c7sc(/C=C8\C(=O)c9cc(C#N)c(C#N)cc9C8=C(C#N)C#N)cc7OC)cc5C6(C)C)c5nsnc35)N=S=N4)cc1C2(C)C] | −5.10 | −4.23 | 1424 | 2.32 | 1430 | 5.97 |
| 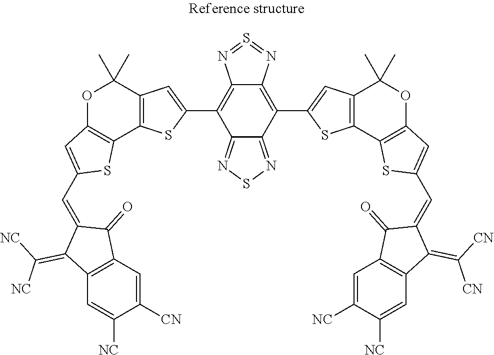 [SMILES: CC1(C)Oc2cc(/C=C3\C(=O)c4cc(C#N)c(C#N)cc4C3=C(C#N)C#N)sc2-c2sc(-c3c4c(c(-c5cc6c(s5)-c5sc(/C=C7\C(=O)c8cc(C#N)c(C#N)cc8C7=C(C#N)C#N)cc5OC6(C)C)c5nsnc35)N=S=N4)cc21] | −5.55 | −4.55 | 1237 | 1.36 | 1269 | 21.6 |
| 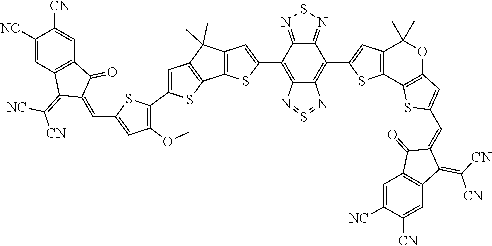 [SMILES: COc1cc(/C=C2\C(=O)c3cc(C#N)c(C#N)cc3C2=C(C#N)C#N)sc1-c1cc2c(s1)-c1sc(-c3c4c(c(-c5cc6c(s5)-c5sc(/C=C7\C(=O)c8cc(C#N)c(C#N)cc8C7=C(C#N)C#N)cc5OC6(C)C)c5nsnc35)N=S=N4)cc1C2(C)C] | −5.32 | −4.38 | 1327 | 1.89 | 1343 | 15.0 |
| 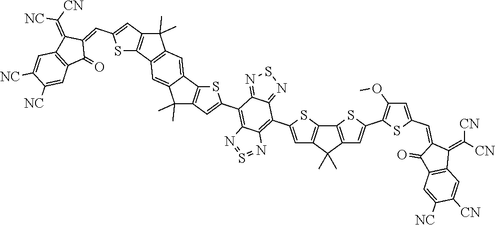 [SMILES: COc1cc(/C=C2\C(=O)c3cc(C#N)c(C#N)cc3C2=C(C#N)C#N)sc1-c1cc2c(s1)-c1sc(-c3c4c(c(-c5cc6c(s5)-c5cc7c(cc5C6(C)C)-c5sc(/C=C6\C(=O)c8cc(C#N)c(C#N)cc8C6=C(C#N)C#N)cc5C7(C)C)c5nsnc35)N=S=N4)cc1C2(C)C] | −5.19 | −4.20 | 1255 | 2.13 | 1321 | 4.6 |

TABLE 8
D¹ and D² are different and B2 are different

| | HOMO/ | LUMO/ | | | | Dipole |
|---|---|---|---|---|---|---|
| Structure/ID | eV | eV | Eg/nm | S1f | Eopt/nm | moment |
| 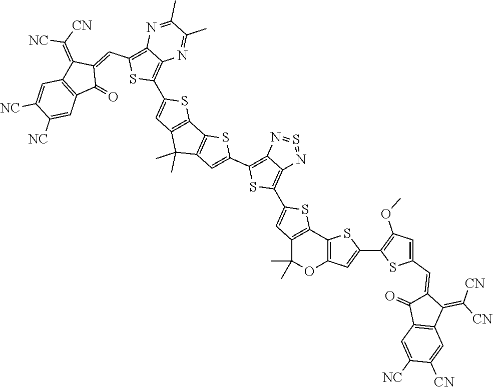 [SMILES: COc1cc(/C=C2\C(=O)c3cc(C#N)c(C#N)cc3C2=C(C#N)C#N)sc1-c1cc2c(s1)-c1sc(-c3sc(-c4cc5c(s4)-c4sc(-c6sc(/C=C7\C(=O)c8cc(C#N)c(C#N)cc8C7=C(C#N)C#N)c7nc(C)c(C)nc67)cc4C5(C)C)c4c3N=S=N4)cc1C(C)(C)O2] | −5.12 | −4.11 | 1231 | 3.24 | 1247 | 10.0 |

1. A compound of formula (I): 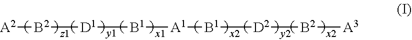
wherein:
A¹ is an electron-accepting group;
D¹ and D² independently in each occurrence is an electron-donating group;
A¹, A² and A³ are each independently an electron-accepting group;
B¹ and B² in each occurrence are independently a bridging group;
x¹ and x² are each independently 0, 1, 2 or 3;
y¹ and y² are each independently at least 1;
z¹ and z² are each independently 0, 1, 2 or 3;
and at least one of (i)-(iv) applies:
(i) (D¹)_{y1} and (D²)_{y2} are different;
(ii) A² and A³ are different;
(iii) (B¹)ₓ₁ and (B¹)ₓ₂ are different; and
(iv) (B²)_{z1} and (B²)_{z2} are different.

2. The compound according to claim 1 wherein D¹ and D² are different.

3. The compound according to claim 1 wherein (B²)_{z1} and (B²)_{z2} are different.

4. The compound according to claim 3 wherein z¹ and z² are different.

5. The compound according to claim 3 wherein z¹ and z² are each 1 and B² of (B²)_{z1} is different from B² of (B²)_{z2}.

6. The compound according to claim 1 wherein A¹ is a group of formula (II): 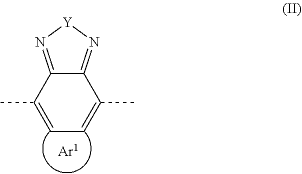
wherein:
Ar¹ is an aromatic or heteroaromatic group; and
Y is O, S, NR⁴ or R¹—C═C—R¹ wherein R¹ in each occurrence is independently H or a substituent wherein two substituents R¹ may be linked to form a monocyclic or polycyclic ring; and R⁴ is H or a substituent.

7. (canceled)

8. The compound according to claim 6 wherein the group of formula (II) has formula (IIb):

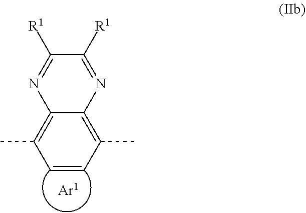

9. The compound according to claim 8 wherein the two R¹ groups are not linked.

10. (canceled)

11. The compound according to claim 8 wherein the two R¹ groups are linked.

12. The compound according to claim 11 wherein the compound of formula (IIb) has formula (IIb-1) or (IIb-2): 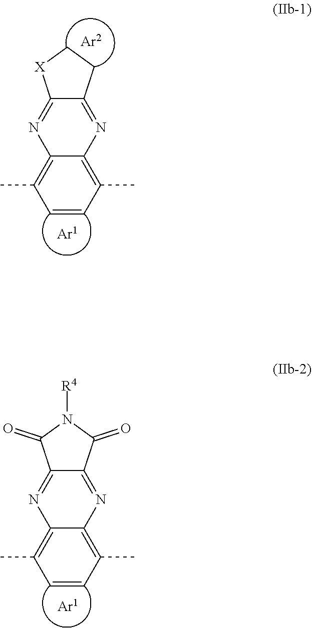
wherein
Ar² is an aromatic or heteroaromatic group which is unsubstituted or substituted with one or more substituents; and
X is selected from O, S, SO₂, NR⁴, PR⁴, C(R³)₂, Si(R³)₂ C═O, C═S and C═C(R⁵)₂ wherein R³ and R⁴ independently in each occurrence are selected from H and a substituent and R⁵ independently in each occurrence is an electron-withdrawing group.

13. (canceled)

14. (canceled)

15. The compound according to claim 1 wherein at least one of z¹ and z² is at least 1 and B² in each occurrence is independently selected from vinylene, arylene, heteroarylene, arylenevinylene and heteroarylenevinylene, each of which is unsubstituted or substituted with one or more substituents.

16. The compound according to claim 1 wherein D¹ and D² are each independently selected from units of formulae (VIIa)-(VIIp): 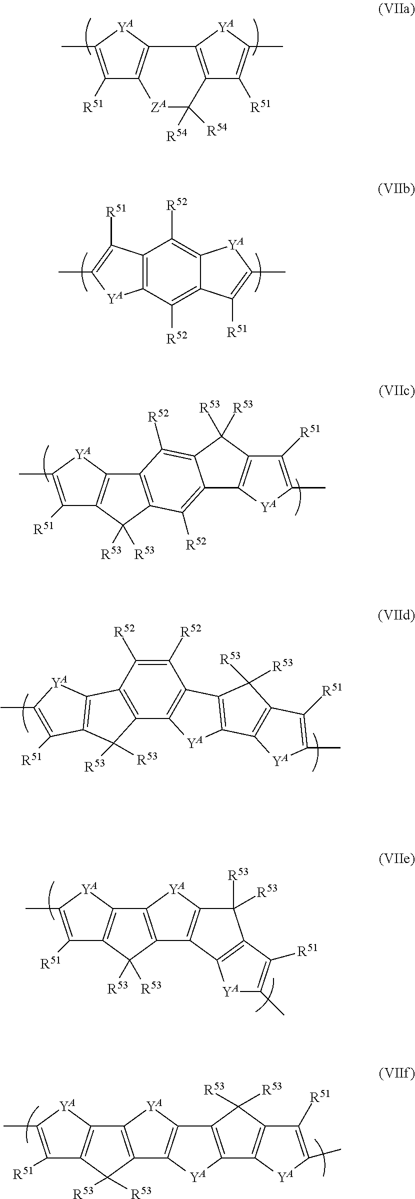 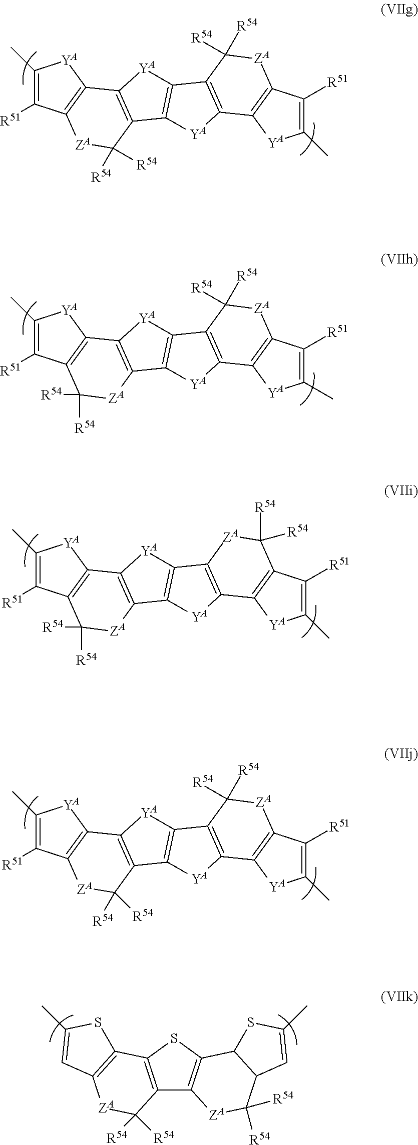 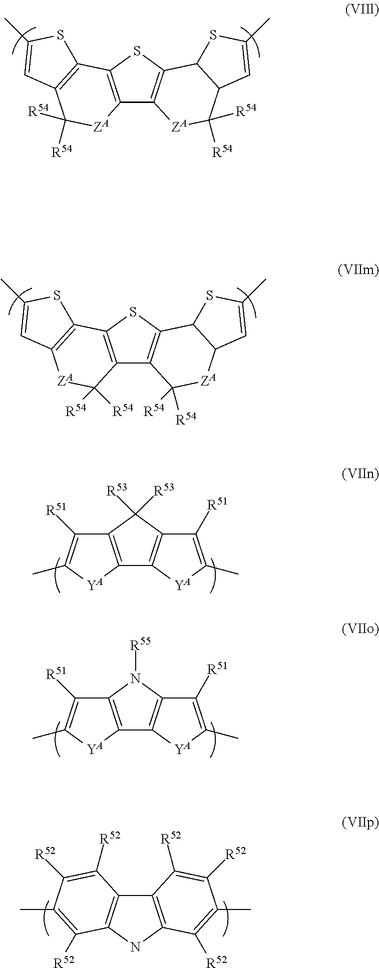
wherein Y^{A} in each occurrence is independently O, S or NR⁵⁵, Z^{A} in each occurrence is O, S, NR⁵⁵ or C(R⁵⁴)₂; R⁵¹, R⁵² R⁵⁴ and R⁵⁵ independently in each occurrence is H or a substituent;
and R⁵³ independently in each occurrence is a substituent.

17. The compound according to claim 1 wherein at least one of A² and A³ comprises a non-aromatic carbon-carbon double bond and a carbon atom of the carbon-carbon double bond is bound directly to D¹ or D² or, if present, to B².

18. (canceled)

19. The compound according to claim 17 wherein at least one of A² and A³ is a group of formula (IIIa-1): 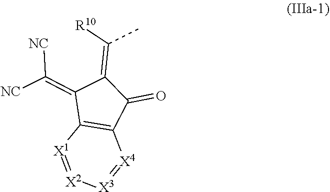
wherein:
each X¹-X⁴ is independently CR¹² or N wherein R¹² in each occurrence is H or a substituent selected from C₁₋₂₀ hydrocarbyl and an electron withdrawing group.

20. The compound according to claim 1 wherein the compound has an absorption peak of greater than 900 nm.

21. A composition comprising an electron-donating material and an electron-accepting material wherein the electron accepting material is a compound according to claim 1.

22. (canceled)

23. An organic photoresponsive device comprising a bulk heterojunction layer disposed between an anode and a cathode and wherein the bulk heterojunction layer comprises a composition according to claim 21.

24. An organic electronic device according to claim 23 wherein the organic photoresponsive device is an organic photodetector.

25. A photosensor comprising a light source and an organic photodetector according to claim 24, wherein the photosensor is configured to detect light emitted from the light source, wherein the light source emits light having a peak wavelength of greater than 900 nm.

26. (canceled)

27. A formulation comprising a compound according to claim 1 dissolved or dispersed in one or more solvents.

28. (canceled)
